(19) United States
(12) Patent Application Publication
(10) Pub. No.: US 2010/0004709 A1
(43) Pub. Date: Jan. 7, 2010

(54) Physiologic stimulation for stroke treatment

(76) Inventor: Hans Alois Mische, (US)

Correspondence Address: Hans A. Mische, 44352 Log Cabin Lane, Grey Eagle, MN, 56336, US

(21) Appl. No.: 12/584,671

(22) Filed: Sep. 10, 2009

Related U.S. Application Data
(63) Continuation-in-part of application No. 11/586,944, filed on Oct. 26, 2006.

(51) Int. Cl. A61N 1/00

(52) U.S. Cl. 607/3

(57) ABSTRACT

Methods and devices are disclosed that provide treatment for neurological strokes and other conditions related to obstructed, hemorrhagic or compromised vascular circulation.

### CROSS REFERENCE TO RELATED APPLICATION

This application is a continuation-in-part of application Ser. No. 11/586,944, filed Oct. 26, 2006. In addition, this application claims the benefit of U.S. Provisional application having Ser. No. 60/259,837, filed Jan. 5, 2001, U.S. Provisional application having Ser. No. 61/191,686, filed Sep. 11, 2008, U.S. Provisional application having Ser. No. ______, filed Nov. 20, 2008, all of which are incorporated herein by reference in their entirety.

### FIELD OF INVENTION

The present invention relates generally to the treatment of neurologic strokes. Methods and devices are disclosed in the context of treating neurologic stroke using physiologic stimulation to control blood flow to and from the brain tissue.

### BACKGROUND OF INVENTION

A number of therapies are available for treating neurological stroke. Drugs and devices are used commonly to attempt to open arteries and veins that are occluded. In some instances, vibrational energy is used to ablate the occlusion causing the stroke. This invention uses vibrational energy, as well as other means of physiologic stimulation (such as electrical stimulation) to initiate the physiologic phenomenon of vasodilation and/or vasoconstriction, modulate the autonomic and afferent nervous systems to activate neuro-physiologic activity in order to re-supply the brain tissue distal of the vessel segment with the occlusion, as well as treat neurological ailments such as depression, pain and movement disorders.

### SUMMARY

The autonomic nervous system (ANS or visceral nervous system) is the part of the peripheral nervous system that acts as a control system functioning largely below the level of consciousness, and controls visceral functions. The ANS affects heart rate, digestion, respiration rate, salivation, perspiration, diameter of the pupils, micturition (urination), and sexual arousal. Whereas most of its actions are involuntary, some, such as breathing, work in tandem with the conscious mind. It is classically divided into two subsystems: the parasympathetic nervous system and sympathetic nervous system. Relatively recently, a third subsystem of neurones that have been named ‘non-adrenergic and non-cholinergic’ neurones (because they use nitric oxide as a neurotransmitter) have been described and found to be integral in autonomic function, particularly in the gut and the lungs. With regard to function, the ANS is usually divided into sensory (afferent) and motor (efferent) subsystems. Within these systems, however, there are inhibitory and excitatory synapses between neurones. The enteric nervous system is sometimes considered part of the autonomic nervous system, and sometimes considered an independent system.

It is known that various frequencies of vibrational energy can cause arteries, veins and capillary beds to dilate or constrict. The terms for such phenomena are vasodilations and vasoconstriction. These phenomena can thus be use to treat victims of neurological strokes. Typically, low frequencies cause dilation while high frequencies cause constriction. Low frequencies generally have a higher average power than high frequencies. Low frequencies tend to travel longer distance with less attenuation than high frequencies.

In the case of hemorrhagic strokes, a ruptured artery results in the lack of distal blood flow to brain tissues.

In the case of an ischemic stroke, a blood clot or other debris such as loose atheroma typically occludes an artery. Sometimes the vessels become occluded due to vasospasm. When the artery is occluded, the blood flow to certain brain tissues is prevented. The lack of blood to the tissue ultimately will result in permanent damage to the tissue and result in neurological function compromise or death.

The current treatment of patients suffering from ischemic strokes include thrombolytic drugs to dissolve clots, mechanical intervention with devices to break up or remove the obstruction, or if the patient arrives to a treatment facility (e.g. hospital) too late for intervention to be of benefit (brain tissue damage is irreversible), than minimum treatment is administered other than rehab for the effects of the stroke. There have been efforts at utilizing ultrasonic and sonic energies to break up the clot or expedite the efficacy of thrombolytics. These technologies have been integrated into intravascular catheter systems or external transducers and have met with safety or effectiveness deficits. The primary intention of this particular invention is to cause controlled and therapeutic vasodilation through the use of vibrational energies to establish blood flow to the regions of the previously fed by the occluded artery.

By utilizing certain frequencies and amplitudes of vibrational energies, arteries, smaller collateral arteries, dormant arteries, or capillary beds can be prompted to expand or open in order to increase, complement or restore blood flow to brain tissues that were fed by the occluded or ruptured artery. Not only can arterial blood flow increase, but brain venous flow can also increase. This can result in a lower capillary bed pressure and allows important oxygenated blood to reach the important brain tissues faster. The flow of cerebrospinal fluids can also be controlled utilizing the same vibrational technologies.

This can be useful in the treatment of ailments such as hydrocephalus and papilledema. Alternatively, certain vibrational frequencies and amplitudes of vibrational energies can cause arteries and/or veins to constrict. These phenomena can be used to reduce or limit the amount of blood flow to certain tissues in order to increase the available blood supply to affected tissues. In this case, the blood supply to the outside of the head, scalp or face can be reduced or limited thus forcing more blood to the brain and the affected tissues. This extra supply may result in an increase of blood pressure within the brain. The increase in pressure may supplement the vibrational energy or alone cause the vessels to expand or be forced open.

A multi-frequency or multi-harmonic source may be utilized to simultaneously obtain vasodilation and vasoconstriction. Typically, low frequencies cause dilation while high frequencies cause constriction. Low frequencies generally have a higher average power than high frequencies. Low frequencies tend to travel longer distance with less attenuation than high frequencies.

Since it is known that lower frequencies have more average power and thus travel farther and deeper than higher frequencies, the lower frequencies could be targeted directly to the brain to expand the deeper vessels and increase flow; higher frequencies can be targeted at the outer portion of the brain, head, and or/face in order to cause constriction of the resident vessels. This scenario thus creates a bi-modal therapy. However, this scenario may be reversed and/or complemented based on clinical need. Selection of frequency and power can also cause the vessels near the brain periphery to expand in order to increase flow to these regions.

Impedance matching the source to the anatomy can also provide beneficial effects and efficiencies.

The vibrational energies can be delivered by various sources. Such sources may be sonic transducers (i.e., sonic, ultrasonic, subsonic, etc), mechanical transducers, electromechanical vibrator, mechanical vibrators, piezoelectric sources, etc), pneumatic, hydraulic, magnetostrictive, or other types that result in the transmission of vibrational energy to the desired target. Hydraulic and pneumatic designs would lend themselves to being used in magnetic resonance imaging (MRI) systems because they could be made without metallic or conductive material that would affect the MRI. This would allow MRI imaging and visualization of the affect of the vibrational energies. In these designs, a pillow like device could be driven hydraulically or pneumatically. The patient's head would rest on the pillow and absorb the vibrational energy during the procedure. The pumps and compressor systems can be located outside of the MRI suite with tubings and conduits running from the systems to the patient site and connected, in one scenario, the pillow. All components can be made of materials that are not attracted to the magnetic energy and that do no affect the MR image. Likewise, the treatment device can be used in conjunction with CAT scan, x-rays, or other imaging and diagnostic equipment. Again, the frequency and amplitude of the desired sources may be designed to gain the desired affect. The frequency range of subsonic to ultrasonic can be used. Likewise, the vibration may be transmitted in a pulsed form or periodically as required. Complex waveforms or combinations of pulses and continuous waveforms may be utilized. These sources can be directly in contact with the external surface if the patients head, face or upper extremities. Likewise, the sources may be utilized in a non-contact fashion such as in the case of sonic transducers. In the addition, the sources may be inserted in the patient near the desired treatment area via the vasculature, conduits, and anatomic openings or surgically with endoscopic techniques as examples.

Other means (electrical stimulation, heat and/or cold, physical manipulation) of stimulating or manipulating the body causes the activation or deactivation of the autonomic and enteric nervous systems and subsequent control of brain, circulatory, respiratory and organ activity. In some cases, an increase in activity is correlated with increase blood flow or physiochemical activity and can be used for therapeutic or diagnostic purposes. In other cases, a reduction in physiologic activity can be exploited to control food digestion in the stomach as an example.

### BRIEF DESCRIPTION OF THE DRAWINGS

Throughout the several views of the drawings several illustrative embodiments of the invention are disclosed. It should be understood that various modifications of the embodiments might be made without departing from the scope of the invention. Throughout the views identical reference numerals depict equivalent structure wherein:

FIG. 1. is a diagram of a patients head residing on a vibrational source

FIG. 2. is a schematic diagram of the head with a discrete vibrational source attached

FIG. 3. is a schematic diagram of a head with a remote transducer directing energy towards it.

FIG. 4. is a schematic diagram of a head within a vibrational therapeutic helmet

FIG. 5. Is a diagram showing the stimulation system connected to a variety of anatomical sites.

### DETAILED DESCRIPTION

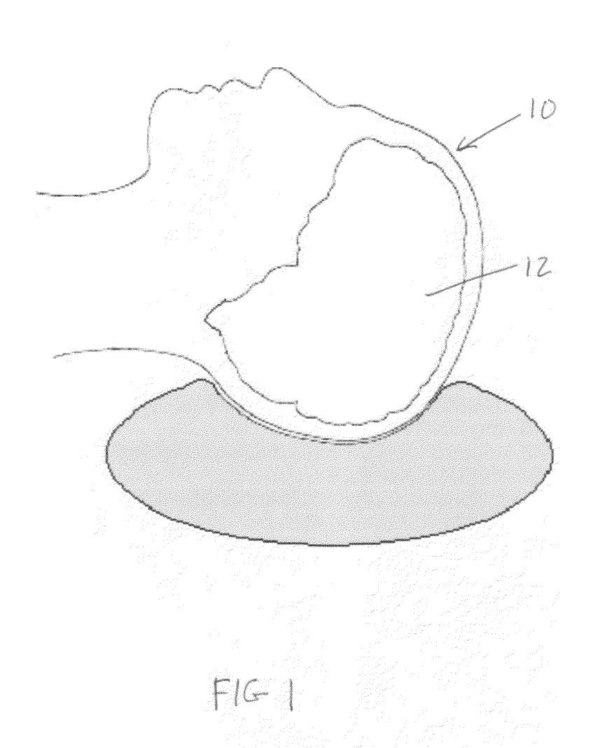

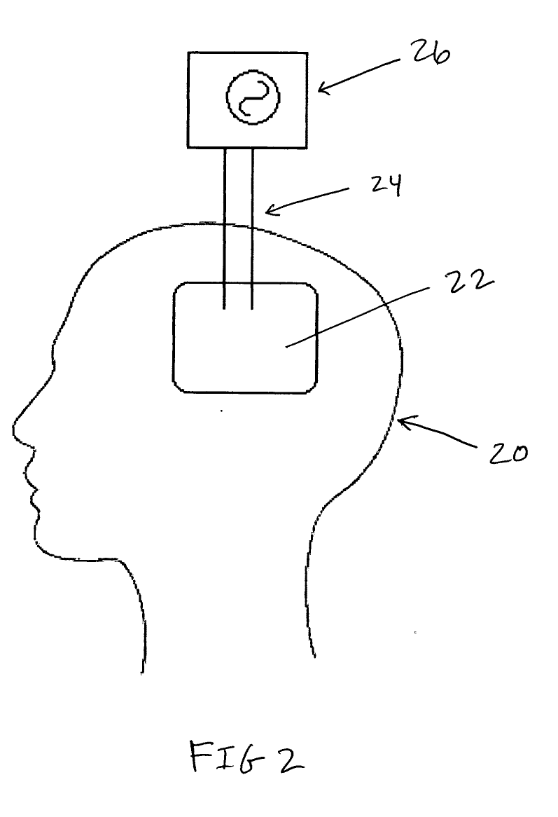

Implementation of the source into various design embodiments is possible. For example, FIG. 1 shows pillow-like device 14 that may be fashioned with a vibration source for convenience, patient comfort, effectiveness and affectivity. A conformable structure would ensure a broader and efficient application of the vibration energy to the head. The pillow could be filled with a fluid or other material in order to get efficient transmission of energy through the patients skull to her brain. Likewise, FIG. 2 shows a design that can be formed into an element 22 that makes contact in an area of the head so as to focus the energy towards specific locations within the head. The vibration source 26 can be built into the element 22 or be, as in this case, a separate component with connections 24 to the element 22. Impedance matching the vibration source to the human anatomy 20 would also be of great benefit for efficiency and/or affectivity. Other means of creating conformable sources are envisioned. The surfaces making contact with the anatomy may consist of materials with varying densities and physical properties in order to optimize and direct energy transmission to desired targets of the anatomy. For example, portions may be fabricated of higher density and low density materials.

Since higher density materials generally transmit vibrational energy better than lower density portions, the higher density portions can be arranged in order to make contact at the desired anatomical locations where vibrational energy transmission is desire. Alternatively, low density portions may be arranged to contact the anatomy where vibrational energy is not desired. This can provide for optimized treatment results as well as patient comfort. Viscoelastic materials can also provide design and treatment options. Another design alternative can use physical gaps or protrusions to direct the energy. Other design alternatives could use more discrete sources for targeted or localized effects. Theses sources could be attached to specific portions or locations of the anatomy in order to achieve the desired affect. The sources could be secured to the patient with straps, glues, tapes, stitched, adhesives, Velcro, magnets, etc.

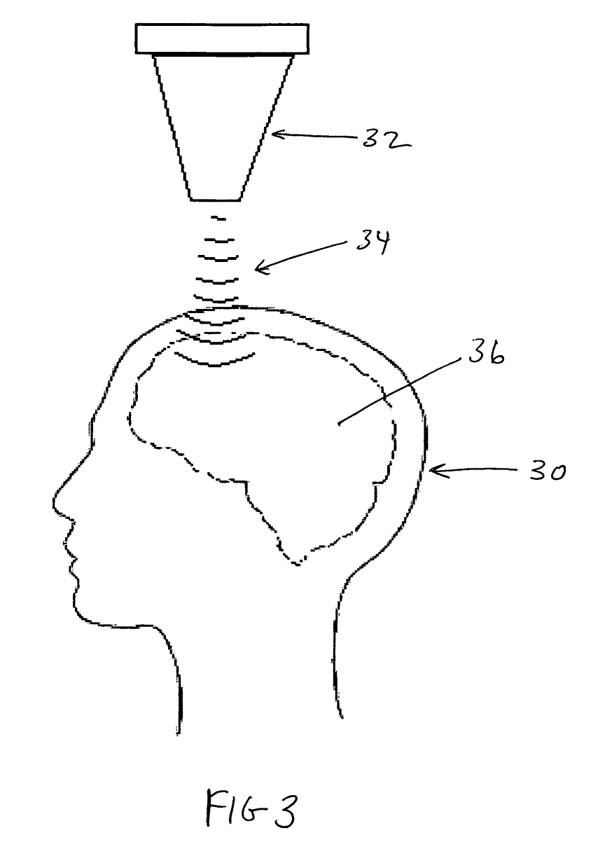

In the remote application of energy, sonic horns or transducers could be aimed at the entire head or certain portions of the head. FIG. 3 shows a potential system that directs vibrational energy 34 to the head 30 via an ultrasonic transducer 32. The vibrational energy is imparted to the brain 36.

The effectiveness of complementary therapeutic drugs, biologics, or other agents may benefits from the application of vibration energy from one of the aforementioned devices. The vibrational energy may help localize the effects, increase the rate and amount of absorption into tissues, or may increase the speed of the effectiveness of thrombolytics or other drugs that can be used to facilitate the treatment of strokes. The application of vibrational energy to the head may also reduce pain and swelling and edema that is associated with strokes. Likewise, the aforementioned devices and methods can also be used to enhance diagnostic imaging procedures such as fluoroscopy, CT, MRI, PET scans. With the application of vibrational energy to various parts of the anatomy, the imaging agents administered to the patient in conjunction with these diagnostic imaging procedures can be distributed more broadly to the desired regions. For example, distal vascular circulatory systems and collateral systems can be identified when the arteries or veins open up and except the imaging agents. Breast exams can be made more effective when imaging agents are more efficiently distributed through the tissue and potential cancerous tumors. During surgical procedures, the vascular anatomy can be identified so as to control bleeding or to spare important vessels from cutting or ligation. The same methods and devices can be modified to assist in the treatment of people suffering from hypothermia or the salvage of frozen limbs by rapidly increasing the circulation of the tissues with oxygenated, warm blood. Likewise, perfusion with blood and oxygen of the anatomy can be made more effective with these same approaches. This can assist in the recovery for victims of poisoning or asphyxiation from toxic gases and substances.

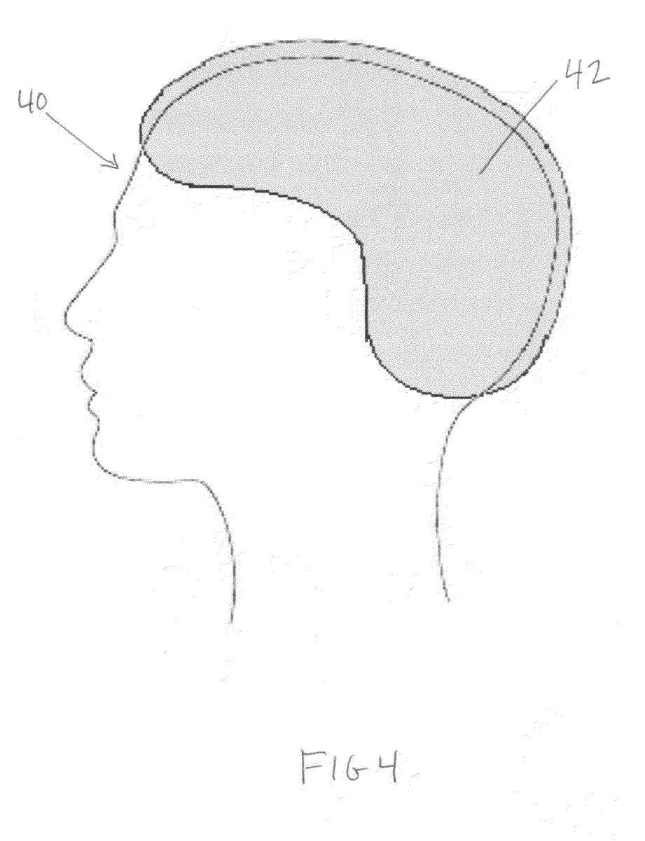

In all cases, the use of these sources may be utilized in emergency settings inside or outside the hospital or in a more controlled environment such as in the ICU, hospital room, catheterization lab, surgical suites, convalescent homes or outpatients facilities. It is envisioned that a version of the source is designed to be battery operated or can be plugged into an ambulance's power source. The battery can be rechargeable. The portability of the source is a benefit for use in emergency situations or for use in ambulance (ground or air). Likewise, portable sources can be placed in public areas such as is the case of placing portable defibrillators in public facilities. The entire source, or portions thereof, can be made to be a single-use, disposable item. A permanent reusable system may also be implemented. Other features may be implemented into the source such as heating, cooling, or electrical stimulation in order to enhance or complement the treatment. The entire therapeutic unit may be contained in a helmet, hat, head band, cuff, wrap, clothing article, bed, gurney, chair, or other structure. FIG. 4 shows a patient 40 wearing a helmet 42 containing a therapeutic unit. The unit can also be incorporated into the patient table of diagnostic imaging such as is used for MRI or CT. In this case, enhanced diagnostic imaging and/or real-time imaging can be performed during therapeutic events. For full-body therapeutic effects, the patient can also be immersed in a fluid bath that transmits vibrational energy. In this case, temperature of the baths can be altered in order to gain various complementary therapeutic effects. Additives to the bath that affect the fluid physical properties such as the density, viscosity, electrical conduction, may provide additional benefits. Low-level electrical stimulation through the bath may also provide analgesic and circulatory benefit.

Other means (electrical stimulation, heat and/or cold, physical manipulation) of stimulating or manipulating the body causes the activation or deactivation of the autonomic and enteric nervous systems and subsequent control of brain, circulatory, respiratory and organ activity. In some cases, an increase in activity is correlated with increase blood flow or physiochemical activity and can be used for therapeutic or diagnostic purposes. In other cases, a reduction in physiologic activity can be exploited to control food digestion in the stomach as an example.

Stimulation (heat, electrical, cold, movement, vibration, etc) of targeted body anatomy causes the stimulation of the afferent nerves in that region of the body (skin, muscles, limbs, face, etc) which initiates an increase in cerebral brain activity, specifically the corresponding cortical activity. The stimulation can be below or above consciousness thresholds. The increase in brain activity is associated with a subsequent increase in the blood flow to that specific region of the brain. The manipulation of afferent nerves at a specific location causes increased blood flow at specific locations of the brain. For example, if the afferent nerves of the hand are stimulated, a specific location of the brain is activated and an increase in blood flow is observed. Likewise, if an arm, leg, or face muscle is stimulated or manipulated, specific locations within the brain are activated, with an associated increase in blood to that brain location.

In the case of treating stroke in a timely and targeted therapeutic fashion, this phenomenon is very important and is the basis for an alternate embodiment of the invention. It is well-known that the brain will suffer permanent damage if blood flow is not restored within a 3 hour timeframe.

At the onset of a neurological stroke, the patient typically exhibits some level of paralysis of the face, arm, or legs. This paralysis is a result of damage to the brain due to the lack of blood flow to the region of the brain that controls the face, arm or leg. In this case, the body is self-diagnostic and informs medical personnel where to stimulate or manipulate the body, affecting the afferent nerve system in order to restore blood flow to the corresponding brain tissue region.

One inventive method of delivery therapeutic medical care would include, but not limited to, the

following steps:
1. Arriving at patient's location.
2. Suspecting the patient has suffered a stroke.
3. Diagnosis the stroke conditions on location via accepted techniques (Cincinnati Stoke Scale, NIH Stroke Scale, etc).
4. Identifying body location exhibiting paralysis or deficit.
5. Attaching the stimulation system to the patient
6. Stimulating or manipulating one or more identified body location to increase blood flow to the corresponding brain region.
7. Transporting patient to healthcare facility for further treatment and care.

This method allows for an early intervention at the patients location when emergency personnel arrive.

In the case of using electrical stimulation, a TENS (transcutaneous electrical nerve stimulator)-type system can be used. A set of electrodes (adhesive, clips, pads, belt, glove, facemask, etc) would be attached to one or more body locations and the stimulator activated. Simultaneous EEG and EKG can be utilized for monitoring purposes and can be incorporated into overall stimulation system.

In this case, the inventive system would include, but not limited to the following:
1. Stimulator system
2. EEG monitor
3. EKG monitor
4. Oxygenation monitoring
5. Respiration monitoring
6. Blood pressure monitoring
7. Glucose monitoring

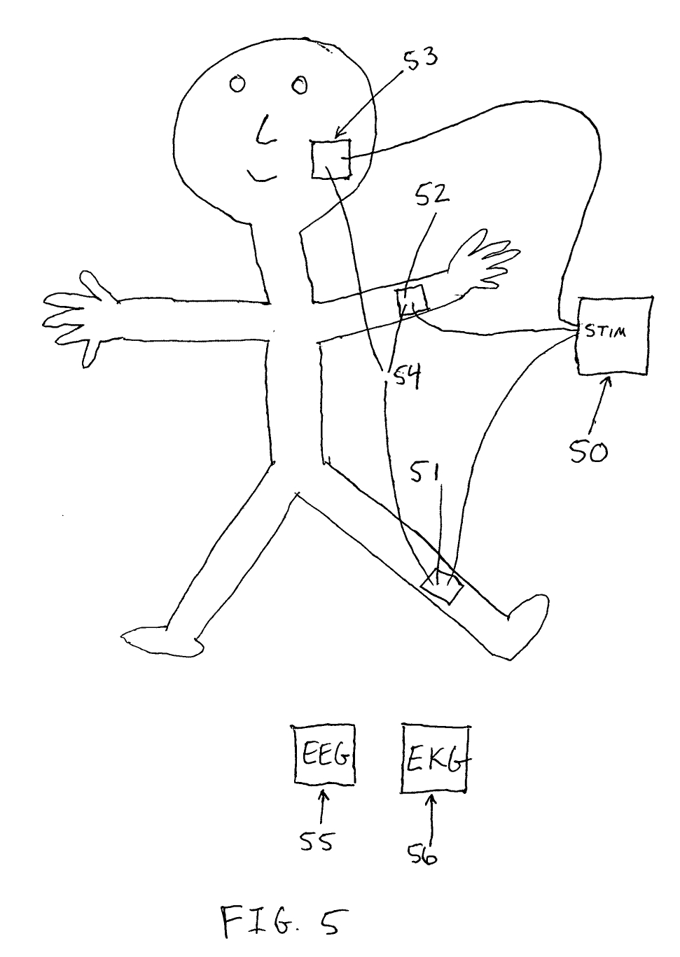

FIG. 5 shows a schematic view of the electrical stimulator system applied to various potential anatomical sites. The stimulator 50 is electrically connected to the leg 51, arm 52, and face 53 via electrodes attachments 54. EEG 55 and EKG 56 monitors can utilize the same electrode attachments 54 for monitoring or their own separate electrodes. All the information from the monitoring system can be transmitted real-time to a physician at a hospital emergency center destination. The stimulator and monitoring system can utilize wire-less or BlueTooth technology to transmit information directly from the patient to the hospital or to on-ambulance equipment. The stimulating system can be incorporated into single-use, disposable designs that are self-contained. For example, the electrical stimulator can be built into an adhesive pad with electrodes, a generator and monitoring features. The inventive system can be organized in a fashion to create a closed-loop system that adjusts the stimulation parameters based on need determined by the monitoring equipment. The level of stimulation may be adjusted and controlled as a function of physiologic blood pressure, cerebral perfusion pressure, intracranial pressure, temperature, respiration rate, oxygen saturation levels. A closed-loop control system may be established with monitoring systems such as an intracranial pressure monitor, EKG, and EEG/cardiac monitor.

The electrodes can be placed proximate to the carotids or other vessels on the neck or head. Other possible locations include ears, lips, tongue, eyes lids/brows, nose, or scalp. In addition, stimulation can be applied to other parts of the anatomy such as the hands, feet, arms, legs, torso, etc. The electrodes can be placed on the skin with pads, clips, needles that penetrate the skin, or probes that enter orifices (e.g. nose, mouth, ear, rectum).

In addition, the electrodes may be incorporated into, or as, belts, bands, helmets, garments, pads, blankets, etc.

One important aspect of the invention is that it can be entirely portable. This would allow the emergency personnel to carry the system into a location to the patient and allow the system to remain with the patient during transport to the hospital, within the hospital, to a long-term care facility and possibly back home if needed for longer term therapy. Other means of targeted stimulation include heat, cold, temperature cycling, vibration, compression, physical movement, acupuncture, prodding, rubbing, massaging, etc. Another means of increasing the blood flow to the brain for stroke and the heart for heart attack victims includes compressing the extremities. Applying external pressure to the lower half of the body helps prevent blood from pooling in the legs. This maintains blood pressure and improves blood flow to the brain, heart and other vital organs. Custom-fitted counter pressure support garments or tourniquet type devices could be used for this.

These could also be integrated into a system that utilizes electrical stimulation and closed looped monitoring and controls.

The source may also contain one or more of EEG, ECG, and Blood Pressure monitoring systems to diagnose and treat the patient. Temperature probes may be installed for internal or external surface measurements in order to correlate to blood flow. IR sensors can also be used to monitor temperatures. Higher body surface temperatures should correlate to increase blood flow, while lower temps would correlate to decreased flow in a certain area of the anatomy. A closed-loop, feedback system can be incorporated in order to optimally adjust vibration frequencies, vibration amplitudes, heat and cooling temperatures, based on output parameters and monitored physiologic parameters. Although this invention is disclosed in the context of treating strokes, the basic principles of the methods and devices may also be used to treat other indications such as peripheral vascular disease or other vasculature, lymphatic systems, tumors, reproductive systems, nervous systems, organs or eyes. It can also be used for cosmetic purposes such as for increasing blood flow through bags under eyes or for bulbous nose syndrome, It can be used for any drug application in order to increase the drug absorption within the tissues or organs by reducing the surface tension of the tissues and cellular matter and matrix by using properly directed vibration energy.

The application of electrical stimulation may also reduce the amount of vasospasm or to treat myocardial infarctions.

Other nerves such as the cranial nerves or the vertebral nerves can be stimulated with the same inventive methodologies to control digestion, heart rate, tongue positioning to treat snoring, movement disorders, appetite, as well as psychological disorders such as depression and anxiety.

What is claimed:

1. A method of treating an acute neurologic stroke comprising the steps of: stimulating the afferent nerves of a body site in order to increase blood circulation in the brain.

2. A method as in claim 1, wherein the stimulating energy affecting the afferent nerves is electricity

3. A method as in claim 1, wherein the stimulating energy affecting the afferent nerves is vibration

4. A method as in claim 1, wherein the stimulating energy affecting the afferent nerves is mechanical

5. A method as in claim 1, wherein the stimulating energy affecting the afferent nerves is heat

6. A method as in claim 1, wherein the stimulating energy affecting the afferent nerves is cold

7. A method as in claim 1, wherein the stimulation energy source is portable

8. A method as in claim 1, wherein the stimulation is used in conjunction with diagnostic equipment

9. A method as in claim 1, wherein the stimulation is used in conjunction with imaging systems

10. A method of treating ischemic neurologic strokes comprising the steps of: diagnosing patients condition, placing electrical leads connected to an electrical stimulator in contact with an anatomical body part, whereby electrical energy stimulates the afferent nerves and results in increased blood flow in the ischemic region of the brain.
